(19) United States
(12) Patent Application Publication
(10) Pub. No.: US 2020/0176114 A1
(43) Pub. Date: Jun. 4, 2020

(54) SYSTEM AND METHOD FOR PROVIDING A LAYER-BASED PRESENTATION OF A MODEL-GENERATED PATIENT-RELATED PREDICTION

(71) Applicant: KONINKLIJKE PHILIPS N.V., EINDHOVEN (NL)

(72) Inventors: OMAR BADAWI, Lutherville Timonium, MD (US); PAMELA JAYNE AMELUNG, CLARKSVILLE, MD (US); SUNIL SWAMI, BALTIMORE, MD (US); XINGGANG LIU, BALTIMORE, MD (US); XI GAO, BALTIMORE, MD (US); Erkan Hassan, Baltimore, MD (US)

(21) Appl. No.: 16/613,007

(22) PCT Filed: May 25, 2018
(86) PCT No.: PCT/EP2018/063787

§ 371 Date: Nov. 12, 2019

Related U.S. Application Data
(60) Provisional application No. 62/512,361, filed on May 30, 2017.

(51) Int. Cl. G16H 50/20; G16H 40/63; A61B 5/00; G16H 50/30

(52) U.S. Cl. CPC G16H 50/20; G16H 40/63; A61B 5/0002; G16H 50/30; A61B 5/7275

(57) ABSTRACT

The present system is configured to provide patient information as input to a prediction model to train the prediction model for generating predictions related to a need for clinical intervention for individual patients. The system is configured to receive vital signs information for one or more vital signs of an individual. The system is configured to process, via the trained prediction model, the vital signs information to generate (i) a first prediction related a clinical intervention need for the individual, (ii) sub-predictions contributing the first prediction or to at least another one of the sub-predictions, and (iii) relatedness information indicating how the first prediction and the contributing sub-predictions are related, the contributing sub-predictions corresponding to respective ones of the weighted predictive parameter features. The system is configured to cause a presentation related to a clinical intervention need for the individual based on the output of the model.

### BACKGROUND

### 1. Field

The present disclosure pertains to a system and method for presenting a model-generated patient related prediction.

### 2. Description of the Related Art

Vital sign threshold-based alert computer systems are known. Typically, such systems generate an alert responsive to the value of a particular monitored vital sign breaching a threshold level, or responsive to the value of the vital sign increasing or decreasing by a predetermined amount over time.

### SUMMARY

Accordingly, one or more aspects of the present disclosure relate to a system configured to provide a layer-based presentation of a model-generated patient-related prediction. The system comprises one or more hardware processors and/or other components. The one or more hardware processors are configured by machine readable instructions to: provide patient information as input to a prediction model to train the prediction model for generating predictions related to a need for clinical intervention for individual patients. The training of the prediction model causes the prediction model to develop weighted predictive parameter features that correspond to patient vital signs, diagnosis information, treatment information, patient demographics (e.g., age, gender, etc.), laboratory information, intensive care unit (ICU) and/or hospital characteristics of the ICU and/or hospital treating the patient, and/or other features. The patient information comprises initial vital signs of patients, treatments provided to the patients with the respective initial vital signs, and respective vital signs resulting from the treatments. The processors are further configured to receive, via one or more sensors, vital signs information for one or more vital signs of an individual; process, via the trained prediction model, the vital signs information to generate (i) a first prediction related a clinical intervention need for the individual, (ii) sub-predictions contributing the first prediction or to at least another one of the sub-predictions, and (iii) relatedness information indicating how the first prediction and the contributing sub-predictions are related, the contributing sub-predictions corresponding to respective ones of the weighted predictive parameter features; cause linking of the first prediction and the contributing sub-predictions based on the relatedness information; and cause, via a user interface, based on the linking, a presentation related to a clinical intervention need for the individual, the presentation comprising the first prediction and the contributing sub-predictions such that user selection related to the first prediction causes display of one or more of the contributing sub-predictions.

By way of a non-limiting example, the present system may be configured to gather data retrospectively and train the model offline. Then that model may be deployed and individual patient may receive predictions generated based on applying the model to their patient specific parameters (e.g., vital signs and other parameters). In some embodiments, the present system may be configured such that the model is retrained in real time.

Another aspect of the present disclosure relates to a method for providing a layer-based presentation of a model-generated patient-related prediction with a prediction system. The system comprises one or more hardware processors configured by machine readable instructions and/or other components. The method comprises: providing patient information as input to a prediction model to train the prediction model for generating predictions related to a need for clinical intervention for individual patients, the training of the prediction model causing the prediction model to develop weighted predictive parameter features that correspond to patient vital signs, diagnosis information, treatment information, patient demographics (e.g., age, gender, etc.), laboratory information, intensive care unit (ICU) and/or hospital characteristics of the ICU and/or hospital treating the patient, and/or other features, the patient information comprising initial vital signs of patients, treatments provided to the patients with the respective initial vital signs, and respective vital signs resulting from the treatments; receiving, via one or more sensors, vital signs information for one or more vital signs of an individual; processing, via the trained prediction model, the vital signs information to generate (i) a first prediction related a clinical intervention need for the individual, (ii) sub-predictions contributing the first prediction or to at least another one of the sub-predictions, and (iii) relatedness information indicating how the first prediction and the contributing sub-predictions are related, the contributing sub-predictions corresponding to respective ones of the weighted predictive parameter features; causing linking of the first prediction and the contributing sub-predictions based on the relatedness information; and causing, via a user interface, based on the linking, a presentation related to a clinical intervention need for the individual, the presentation comprising the first prediction and the contributing sub-predictions such that user selection related to the first prediction causes display of one or more of the contributing sub-predictions.

Still another aspect of present disclosure relates to a system configured to provide a layer-based presentation of a model-generated patient-related prediction. The system comprises: means for providing patient information as input to a prediction model to train the prediction model for generating predictions related to a need for clinical intervention for individual patients, the training of the prediction model causing the prediction model to develop weighted predictive parameter features that correspond to patient vital signs, diagnosis information, treatment information, patient demographics (e.g., age, gender, etc.), laboratory information, intensive care unit (ICU) and/or hospital characteristics of the ICU and/or hospital treating the patient, and/or other features, the patient information comprising initial vital signs of patients, treatments provided to the patients with the respective initial vital signs, and respective vital signs resulting from the treatments; means for receiving, via one or more sensors, vital signs information for one or more vital signs of an individual; means for processing, via the trained prediction model, the vital signs information to generate (i) a first prediction related a clinical intervention need for the individual, (ii) sub-predictions contributing the first prediction or to at least another one of the sub-predictions, and (iii) relatedness information indicating how the first prediction and the contributing sub-predictions are related, the contributing sub-predictions corresponding to respective ones of the weighted predictive parameter features; means for causing linking of the first prediction and the contributing sub-predictions based on the relatedness information; and means for causing, via a user interface, based on the linking, a presentation related to a clinical intervention need for the individual, the presentation comprising the first prediction and the contributing sub-predictions such that user selection related to the first prediction causes display of one or more of the contributing sub-predictions.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1 illustrates a system configured to facilitate patient-related predictions via a prediction model and presentation thereof.

FIG. 2A illustrates an amount of variability in a vital sign over time, a value of the vital sign at a given point in time, and a slope of the values of the vital sign over time.

FIG. 2B illustrates the value of the vital sign relative to a baseline range for the vital sign, with the default values generated by a historical population mean plus and minus one population standard deviation for the vital sign in a similar population.

FIG. 2C illustrates a combination of a prediction for the current vital sign based on prior data, the upper and lower bounds of the confidence interval for the prediction, and an amount the current vital sign value deviates from the expected value of the vital sign.

FIG. 3 is a schematic illustration of training a predictive model.

FIG. 4 illustrates a risk score presented to caregivers and/or other users that indicates the probability and or risk of requiring a clinical intervention for an individual.

FIG. 5 illustrates a method for providing a layer-based presentation of a model-generated patient-related prediction.

### DETAILED DESCRIPTION OF EXEMPLARY EMBODIMENTS

As used herein, the singular form of “a”, “an”, and “the” include plural references unless the context clearly dictates otherwise. As used herein, the term “or” means “and/or” unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

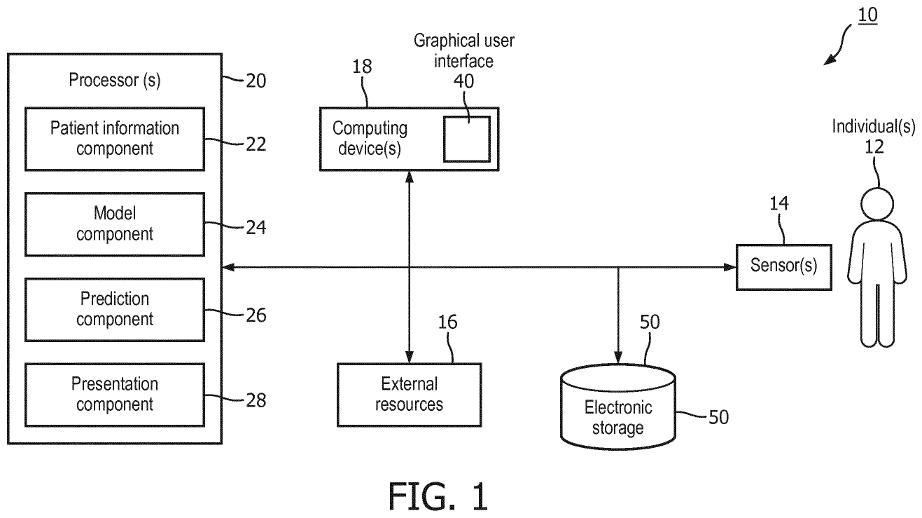

FIG. 1 is a schematic illustration of a system 10 configured to facilitate patient-related predictions via a prediction model and presentation thereof. In some embodiments, system 10 is configured to identify hospitalized patients who are likely to require an intervention and present the information in a form that is clinically meaningful and supports the decision-making process for caregivers (e.g., doctors, nurses, clinicians, family members, friends, etc.). System 10 enables clinicians, for example, and/or other users to efficiently prioritize patients by their probability of need for intervention and easily interpret the rationale for the estimated probability. It should be noted that, although the present disclosure refers to predicting a need for (e.g., acute) intervention in hospitalized (e.g., intensive care unit (ICU)) patients, this is not intended to be limiting. The system and method described herein may apply to any patient-related prediction or other predictions. For example, system 10 may determine the probability of: 1) survival; 2) successful extubation from a ventilator; 3) developing a complication such as acute kidney injury or delirium; and/or other predictions.

Although computer-assisted patient alert and prediction systems exist, output from such typical systems tends to be difficult to interpret as a user may not be able to intuitively understand how the output was generated. As an example, if a given model-generated risk estimate is generated, it may be difficult for the user to understand how the risk estimate was generated, or why one patient has a higher risk indicator than another patient. In some embodiments, system 10 displays an indication of risk or other prediction that is easy to understand for any given patient or comparison of multiple patients. Additionally, or alternatively, typical systems often define alerting and prediction criteria regardless of individual patient characteristics (e.g., by generating alerts and predictions based on population level data without considering the characteristics of the individual patients) or require manual adjustment of threshold levels for individual patients. In some embodiments, system 10 automatically considers specific vital sign trends for individual patients when determining a patient's risk of needing intervention. In some embodiments, system 10 automatically adjusts threshold levels for individual patients based on the specific vital sign trends of the respective patients or other information.

In typical systems, criteria for activation of an alert are often arbitrary. For example, if a heart rate alert is set to activate at or above 150 beats per minute (bpm), then this implies a heart rate of 149 bpm is a significantly lower risk than 150 bpm (when in fact the difference in a 149 versus 150 bpm heart rate is very small). Users may be able to modify the threshold in a typical system, but any threshold modification would also be arbitrary. In some embodiments, system 10 overcomes this limitation by determining a risk indicator (e.g., a numeric risk score) which may be used to rank patients on a continuous scale (e.g., which is not arbitrarily cut off at 150 bpm). Additionally, or alternatively, an arbitrary threshold may be used for alerting—however, patients just below the designated threshold will be identifiable, and the relative risk compared to other patients who may be just above the threshold is intuitive to doctors (for example) viewing the displayed indication (e.g., because, as described herein, the risk indicator (a numeric risk score for example) may be a number on a continuous number line, such as 1-100 for example, rather than a red or yellow light, or merely an alarm sound as in prior art systems).

Typical systems have minimal ability to prioritize one patient over another in a meaningful way. For example, typical systems merely rank patients in order of most recent alert. As another example, in typical systems, any two patients meeting low blood pressure alert criteria will activate similar alerts, even if one patient has a much lower blood pressure than another. Similarly, if one patient also has an abnormal heart rate or respiratory rate, such information would cause an independent alert, which would not be aggregated to raise a treatment risk priority of the patient. Also, typical systems have several different individual alert types (e.g., a heart rate alert, a blood pressure alert, a respiratory rate alert, an oxygen saturation alert, etc.) with multiple subtypes (e.g., trend, threshold, high, low, severe, moderate, etc. as described above). The wide range of physiologic monitoring parameters and alerts can yield a high alarm rate. The high alarm rate may cause alert fatigue (e.g., ignoring alerts, downplaying their importance, etc.) in doctors or nurses (for example) caring for a patient.

In contrast, in some embodiments, system 10 generates an aggregated output by combining vital signs information for a patient and assigning weights to vital sign feature parameters (described below) appropriately by risk for a given vital sign. For example, a patient with low blood pressure, high heart rate, and high respiratory rate would activate (at least) three different alerts in a typical system. In some embodiments, these vital sign levels would generate a single indication (e.g., a score) which would help a doctor (for example) prioritize the patient over one with a similarly high heart rate but normal blood pressure and respiratory rate.

As described above, current alert systems may have default threshold values which can be manually tailored for a given patient. This often creates difficulty when sub-populations of patients require the same unique considerations, which would require a user to perform the same manual tailoring for each patient in the sub-population. For example, patients admitted to a mixed ICU after coronary artery bypass grafting (CABG) surgery may have unique physiology that is different than physiology of patients admitted for heart failure. In some embodiments, system 10 is configured such that users can more effectively and efficiently care for these patients because default alert threshold values and/or other system parameters can be managed at a group level, rather than requiring manual customization for individual patients.

In some embodiments, system 10 is configured to, at a given time, generate a comprehensive indicator (e.g., a score) for monitored patients based on comprehensive vital sign information for a given patient. The indicator represents a need for urgent attention in the patient. In some embodiments, system 10 includes one or more of sensors 14, external resources 16, computing devices 18, processors 20, electronic storage 50, and/or other components. It should be noted that a single individual 12 is illustrated in FIG. 1, but this is not intended to be limiting. System 10 is configured to operate as described herein for any number of individuals 12.

Sensors 14 are configured to generate output signals that convey information related to the vital signs and/or other physiological information of individual 12. In some embodiments, the output signals from sensors 14 are received by processor 20 (described below) and/or other components of system 10. In some embodiments, sensors 14 include but are not limited to equipment used in hospitals, doctor's offices, and/or other medical facilities to monitor vital signs and/or other physiological information (e.g., pulse rate monitors, blood (e.g., arterial) pressure monitors, blood oxygenation monitors, glucose monitors, respiration monitors, weight scales, thermometers, electrocardiogram (EKG) equipment), test equipment (e.g., imaging equipment such as an MRI and/or an x-ray machine, an ultrasound, electroencephalogram (EEG) equipment, etc.), equipment for treating patients and/or individual(s) 12 (e.g., respirators/ventilators, light therapy devices, etc.), or other devices. In some embodiments, sensors may be included in desktop computers, laptop computers, tablet computers, smartphones, cameras, video equipment, wearable devices, etc., associated with individual(s) 12, and/or other devices. In some embodiments, sensors 14 include at least one cardiovascular sensor (e.g., a heart rate sensor, a mean arterial pressure sensor, etc.) and at least one respiratory sensor (e.g., a breathing rate sensor). In some embodiments, sensors 14 are operatively coupled to individual 12. Operative coupling may include any form of removable and/or fixed attachment to individual 12 that facilitates wireless and/or wired transmission of the output signals from sensors 14. By way of several non-limiting examples, operative coupling may include a blood pressure cuff that is temporarily coupled to the arm of individual 12 using a Velcro strap; a blood oxygen monitor that is clipped to the finger of individual 12; a respiration sensor that is fixedly coupled to a mask worn by individual 12; and/or other methods of coupling sensors 14 to individual 12. In some embodiments, sensors 14 and/or operations performed by sensors 14 may include capturing data by interfacing with a hospital electronic health record (HER) and/or lab system. This could include capturing data related to and/or include patient demographics, diagnosis, treatments, physiology, lab values, and/or other information.

External resources 16 include sources of patient and/or other information. In some embodiments, external resources 16 include sources of patient and/or other information such as databases, websites, etc.; external entities participating with system 10 (e.g., a medical records system of a health care provider that stores medical history information for populations of patients), one or more servers outside of system 10, a network (e.g., the internet), electronic storage, equipment related to Wi-Fi technology, equipment related to Bluetooth® technology, data entry devices, sensors, scanners, and/or other resources. For example, in some embodiments, external resources 16 may include a database where medical history information for a plurality of patients similar to individual 12 and/or other patients are stored, and/or other sources of information such as sources of information related to patient demographics, diagnoses, problem lists, treatments, lab data, and/or other information. In some embodiments, the patient information comprises initial vital signs of patients, treatments provided to the patients with the respective initial vital signs, respective vital signs resulting from the treatments, and/or other information. In some implementations, some or all of the functionality attributed herein to external resources 16 may be provided by resources included in system 10. External resources 16 may be configured to communicate with processor 20, computing devices 18, sensors 14, electronic storage 50, and/or other components of system 10 via wired and/or wireless connections, via a network (e.g., a local area network and/or the internet), via cellular technology, via Wi-Fi technology, and/or via other resources.

Computing devices 18 are configured to provide interfaces between caregivers (e.g., doctors, nurses, friends, family members, etc.), individual 12, and/or other users, and system 10. In some embodiments, individual computing devices 18 are and/or are included in desktop computers, laptop computers, tablet computers, smartphones, and/or other computing devices associated with individual caregivers, individual 12, and/or other users. In some embodiments, individual computing devices 18 are, and/or are included in equipment used in hospitals, doctor's offices, and/or other medical facilities to monitor individual 12; test equipment; equipment for treating individual 12; data entry equipment; and/or other devices. Computing devices 18 are configured to provide information to and/or receive information from the caregivers, individual 12, and/or other users. For example, computing devices 18 are configured to present a graphical user interface 40 to the caregivers to facilitate display of a risk score and/or other information for individual 12 (e.g., as described below). In some embodiments, graphical user interface 40 includes a plurality of separate interfaces associated with computing devices 18, processor 20 and/or other components of system 10; multiple views and/or fields configured to convey information to and/or receive information from caregivers, individual 12, and/or other users; and/or other interfaces.

In some embodiments, computing devices 18 are configured to provide graphical user interface 40, processing capabilities, databases, and/or electronic storage to system 10. As such, computing devices 18 may include processors 20, electronic storage 50, external resources 16, sensors 14, and/or other components of system 10. In some embodiments, computing devices 18 are connected to a network (e.g., the internet). In some embodiments, computing devices 18 do not include processors 20, electronic storage 50, external resources 16, sensors 14, and/or other components of system 10, but instead communicate with these components via the network. The connection to the network may be wireless or wired. For example, processor 20 may be located in a remote server and may wirelessly cause display of graphical user interface 40 to the caregivers on computing devices 18. As described above, in some embodiments, an individual computing device 18 is a laptop, a personal computer, a smartphone, a tablet computer, and/or other computing devices. Examples of interface devices suitable for inclusion in an individual computing device 18 include a touch screen, a keypad, touch sensitive and/or physical buttons, switches, a keyboard, knobs, levers, a display, speakers, a microphone, an indicator light, an audible alarm, a printer, and/or other interface devices. The present disclosure also contemplates that an individual computing device 18 includes a removable storage interface. In this example, information may be loaded into a computing device 18 from removable storage (e.g., a smart card, a flash drive, a removable disk) that enables the caregivers, individual 12, and/or other users to customize the implementation of computing devices 18. Other exemplary input devices and techniques adapted for use with computing devices 18 include, but are not limited to, an RS-232 port, RF link, an IR link, a modem (telephone, cable, etc.) and/or other devices.

Processor 20 is configured to provide information processing capabilities in system 10. As such, processor 20 may comprise one or more of a digital processor, an analog processor, a digital circuit designed to process information, an analog circuit designed to process information, a state machine, and/or other mechanisms for electronically processing information. Although processor 20 is shown in FIG. 1 as a single entity, this is for illustrative purposes only. In some embodiments, processor 20 may comprise a plurality of processing units. These processing units may be physically located within the same device (e.g., a server), or processor 20 may represent processing functionality of a plurality of devices operating in coordination (e.g., one or more servers, one or more computing devices 18 associated with caregivers, a piece of hospital equipment, sensors 14, devices that are part of external resources 16, electronic storage 50, and/or other devices.)

In some embodiments, processor 20, sensors 14, external resources 16, computing devices 18, electronic storage 50, and/or other components may be operatively linked via one or more electronic communication links. For example, such electronic communication links may be established, at least in part, via a network such as the Internet, and/or other networks. It will be appreciated that this is not intended to be limiting, and that the scope of this disclosure includes embodiments in which these components may be operatively linked via some other communication media. In some embodiments, processor 20 is configured to communicate with sensors 14, external resources 16, computing devices 18, electronic storage 50, and/or other components according to a client/server architecture, a peer-to-peer architecture, and/or other architectures.

As shown in FIG. 1, processor 20 is configured via machine-readable instructions to execute one or more computer program components. The one or more computer program components may comprise one or more of a patient information component 22, a model component 24, a prediction component 26, a presentation component 28, and/or other components. Processor 20 may be configured to execute components 22, 24, 26, and/or 28 by software; hardware; firmware; some combination of software, hardware, and/or firmware; and/or other mechanisms for configuring processing capabilities on processor 20.

It should be appreciated that although components 22, 24, 26, and 28 are illustrated in FIG. 1 as being co-located within a single processing unit, in embodiments in which processor 20 comprises multiple processing units, one or more of components 22, 24, 26, and/or 28 may be located remotely from the other components. The description of the functionality provided by the different components 22, 24, 26, and/or 28 described below is for illustrative purposes, and is not intended to be limiting, as any of components 22, 24, 26, and/or 28 may provide more or less functionality than is described. For example, one or more of components 22, 24, 26, and/or 28 may be eliminated, and some or all of its functionality may be provided by other components 22, 24, 26, and/or 28. As another example, processor 20 may be configured to execute one or more additional components that may perform some or all of the functionality attributed below to one of components 22, 24, 26, and/or 28.

In some embodiments, patient information component 22 is configured to obtain patient information. The patient information indicates health information for a plurality of patients and/or other information. The health information may be related to vital signs of the plurality of patients, physical conditions experienced by the plurality of patients, medical records describing previous medical treatment provided to the plurality of patients, and/or other information. For example, the patient information may comprise initial vital signs of patients, treatments provided to the patients with the respective initial vital signs, respective vital signs resulting from the treatments, and/or other information.

Patient information component 22 is configured to obtain the patient information from external resources 16 (e.g., one or more external databases included in external resources 16), electronic storage 50 included in system 10, one or more sensors 14, and/or other sources of information. For example, the patient information may be obtained from an electronic ICU (eICU) platform and/or other sources.

In some embodiments, an eICU platform is a telehealth software application, designed to provide clinicians with a set of tools that display organized clinical information and highlight interrelationships. Used by an integrated team of bedside and remote clinicians, the system helps facilitate the delivery of high quality in-hospital care and supports rapid recognition of potential patient problems. This comprehensive view of a patients' clinical status supports timely decision-making and effective communication and coordination among the care team, while helping to plan, document and standardize care around best practices.

In some embodiments, the patient information is anonymized (e.g., processed to remove any information that might identify a particular patient) by patient information component 22 and stored in a relational database (e.g., such as an electronic research institute (eRI) database) and/or other databases. In some embodiments, patient information component 22 is configured to perform natural language processing on the patient information, and generate variable files for vital signs (such as heart rate, blood pressure, etc.) and clinically relevant (to the particular treatment received by a given patient) interventions and/or events (e.g., such as blood infusions, albumin usage, provision of a fluid bolus, vasoactive usage, death, etc.). In some embodiments, patient information component 22 uses a sliding window technique to scan heart rate (HR), mean arterial pressure (MAP), and/or other vital sign information (these two examples are not intended to be limiting) through 60 minute, 180 minute, and/or other windows of time for a duration of a patient's stay in a medical facility, respectively. The 60 minute and 180 minute durations described above are only examples and are not intended to be limiting. Other windows of time may be used. In some embodiments, patient information component 22 is configured to combine and/or correlate the information from the clinically relevant interventions and/or events with the vital signs from the corresponding windows of time and generate consensus categorical outcome determinations (e.g., whether the patient was stable, unstable, status unable to be determined, etc.). In some embodiments, these features (vital signs, clinically relevant interventions and/or events) and outcomes (stable, unstable, unknown) comprise input for a prediction model (described below).

In some embodiments, model component 24 is configured to provide the patient information to the prediction model. As described above, the patient information comprises information related to initial vital signs of patients, treatments provided to the patients with the respective initial vital signs, respective vital signs resulting from the treatments (e.g., the features (vital signs, clinically relevant interventions and/or events) and outcomes (stable, unstable, unknown)), and/or other information. In some embodiments, the prediction model is a logistic regression model and/or other models. The patient information is provided to the prediction model to train the prediction model for generating predictions related to a need for clinical intervention for individual patients and/or for other purposes. In some embodiments, model component 24 is configured such that the logistical regression model (or other statistical model) is trained and validated using cross-validation and/or other techniques. In some embodiments, model component 24 is configured such that the predictive model is designed with a set of features specifically used due to their clinically relevant distinctions. For example, a decreasing blood pressure will likely indicate a higher risk for need of fluid resuscitation if the patient has a low blood pressure, rather than a high blood pressure. The predictive model is designed to account for these clinically meaningful groups by creating unique coefficients for a wide variety of clinical combinations. In some embodiments, system 10 is designed to be configurable so new groups and/or new weights can be introduced as models evolve.

In some embodiments, the training and/or validation of the prediction model causes the prediction model to develop weighted predictive parameter features that correspond to patient vital signs and/or other patient information. For example, model component 24 is configured such that weighted predictive parameter features corresponding to vital signs, trends in vital signs, particular medical facilities (e.g., different types of ICU's), types of patients (e.g., a heart failure patient versus a CABG patient), and/or other patient information are determined. In some embodiments, model component 24 may be configured such that a given predictive parameter feature is a combination of two or more other weighted predictive parameter features.

In some embodiments, model component 24 is configured such that individual weights (e.g., coefficients) are determined for individual predictive parameter features. In some embodiments, the individual weights are determined based on a type of patient information associated with the predictive parameter features and/or other information. Continuing with the example above, different weights may be determined for predictive parameter features corresponding to different vital signs and/or trends in vital signs, different weights may be determined for predictive parameter features corresponding to particular medical facilities (e.g., different types of ICU's), different weights may be determined for predictive parameter features corresponding to different types of patients (e.g., a heart failure patient versus a CABG patient), and/or other weighting may be determined. In some embodiments, model component 24 is configured such that individual weights are manually entered and/or selected (e.g., via a computing device 18) by caregivers and/or other users.

In some embodiments, model component 24 is configured such that the weighted parameters are developed by training a model to identify near term interventions in patients and identify the weights for parameters predictive of these outcomes. Similar outcomes are grouped together (e.g., bolus of fluids and initiation of vasopressors) to create a model which can complement other models targeting other outcomes such as need for intubation or ventilator support. The clinical relevance of the groupings allow for different weights to be derived in meaningful ways which will be intuitive for clinicians.

By way of a non-limiting example, in some embodiments, the weighted predictive parameter features include an amount of variability in a given vital sign, a slope for the given vital sign determined based on values of the given vital sign over time, an amount of deviation from a baseline value and/or range (e.g., baseline heart rate is between about 70 and about 90 beats per minute) baseline range for the given vital sign, an amount of deviation from an expected level of the given vital sign for the monitored individual, and/or other features. In some embodiments, system 10 may be configured such that individual patients have a baseline value for a vital sign and/or some other parameter, but the system is configured to default the algorithm to using a population derived value (e.g., as described herein). In some embodiments, system 10 may be configured such that users are able to modify and/or adapt a patient specific baseline as well as the baseline for sub-populations of their choice (e.g., all heart failure patients, all male heart failure patients above 65 years of age, etc.). In some embodiments, system 10 may determine a patient specific baseline based on available previously recorded data.

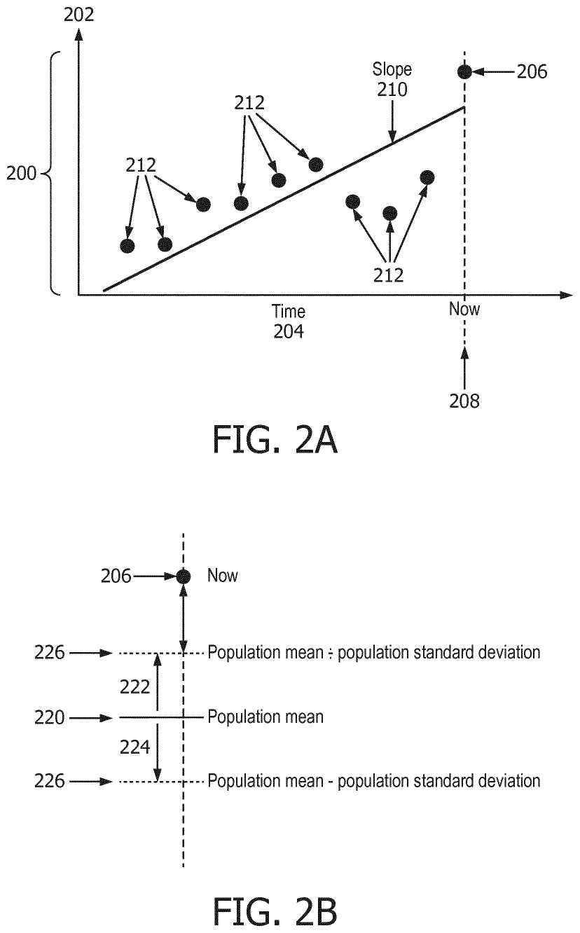

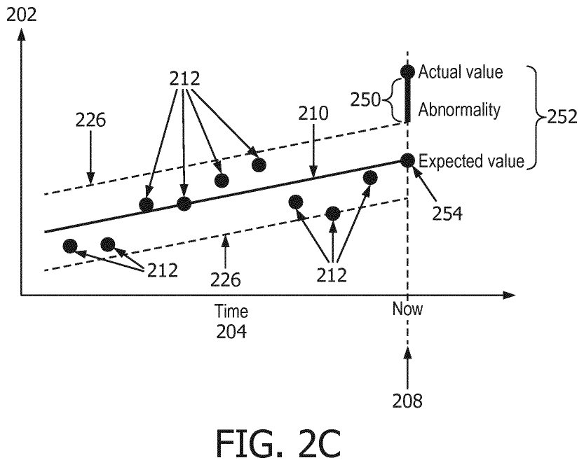

As an example, at least one of the predictive parameter features may be a combination of the slope for the given and the amount of deviation from the baseline range for the given sign, a combination of the amount of deviation from the baseline range for the given vital sign and the amount of deviation from the expected level of the given vital sign for the monitored individual, other combinations, or a feature generated from one or more of the foregoing combinations. Example weighted predictive parameter features are illustrated in FIG. 2A-FIG. 2C. FIG. 2A illustrates an amount of variability 200 in a vital sign 202 over time 204, a value 206 of vital sign 202 at a given point 208 in time (“now”), and a slope 210 of the values 212 of vital sign 202 over time 204. FIG. 2B illustrates value 206 of vital sign 202 (FIG. 2A) relative to a baseline range 220 for vital sign 202, and relative to baseline range 220 plus 222 and minus 224 one population standard deviation (used as an example only and is not intended to be limiting) 226 for vital sign 202. Baseline range 220 and/or population standard deviations 226 may be for the population of patients whose patient data was obtained by patient information component 22 described above related to FIG. 1, and/or other populations. FIG. 2C illustrates a combination of slope 210 for vital sign 202, an amount of deviation 250 from baseline range 220 plus one standard deviation 226 for vital sign 202, and an amount of deviation 252 from an expected level 254 of vital sign 202.

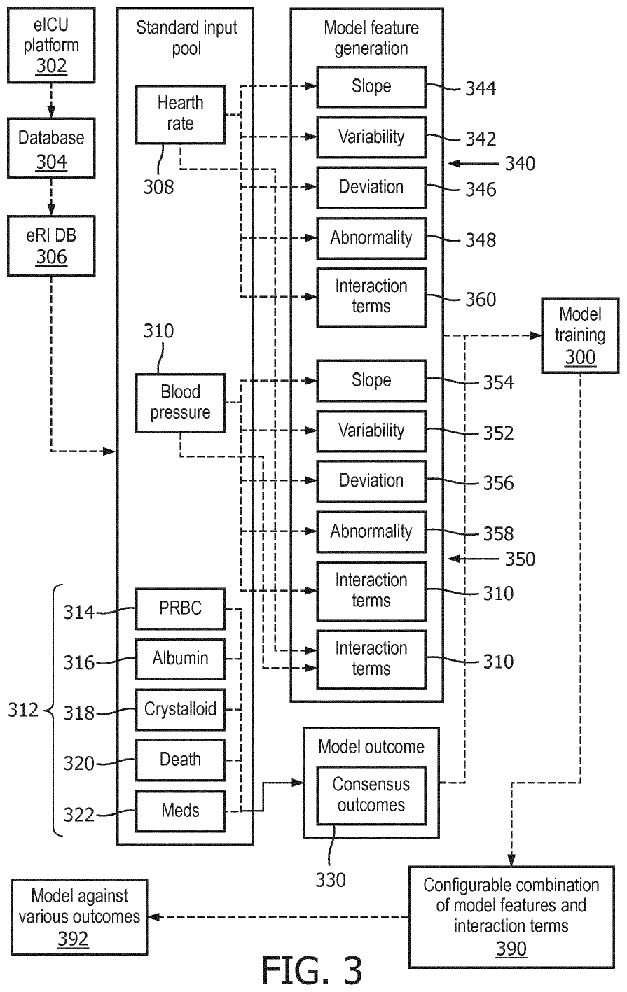

FIG. 3 is a schematic illustration of training 300 the predictive model using patient information (including the vital signs and clinically relevant interventions and/or events), weighed predictive parameter features described above, interaction terms, or other information. In some embodiments, interaction terms include the ability to stratify weights by clinically meaningful groups. For example, if there are five defined categories of blood pressure deviation from baseline and five categories for the trend/slope of the patient's blood pressure, then there would be 25 possible unique combinations when these parameters are ‘interacted’ in a regression model. In this scenario, each unique combination will receive a unique weight relative to the risk associated with a patient being in that group. Furthermore, these interaction terms can continue to expand as is clinically useful and is supported by available data. For example, the above example can also be interacted with unique combinations of five (or other numbers) categories of heart rate trends/slope. Addition of this interaction term would create 125 possible unique combinations of heart rate slope, blood pressure deviation from baseline and blood pressure slope. Each of these will create highly specific weights for patients falling into these categories and thereby increase the clinical accuracy, relevance, and interpretation of the model output.

For example, as shown in FIG. 3, patient information may be obtained from an eICU platform 302 and stored in a database 304. The patient information may be anonymized and stored in a relational database 306. In some embodiments, the patient information is processed using natural language processing to generate variable files for vital signs (such as heart rate 308, blood pressure 310, etc.) and clinically relevant interventions and/or events 312 (e.g., such as packed red blood cell (PRBC) infusion 314, albumin usage 316, crystalloid 318, death 320, provision of medication 322, etc.). In some embodiments, the information from the clinically relevant interventions and/or events are combined and/or correlated with the vital signs from the corresponding windows of time to generate consensus categorical outcome determinations 330. The prediction model may develop and/or validate (e.g., after training) weighted predictive parameter features 340 (e.g., corresponding to a first vital sign and/or other characteristics of a patient) and 350 (e.g., corresponding to a second vital sign and/or other characteristics of a patient), interaction terms 360 (e.g., corresponding to the first vital sign type and/or other characteristics) and 370 (e.g., corresponding to the second vital sign type and/or other characteristics), and/or other information that correspond to patient vital sign types (the example of two different sets of features is not intended to be limiting). As shown in FIG. 3, there may be more than one interaction term and/or set of interaction terms 370 that correspond to a given vital sign type. As described above, in some embodiments, the weighted predictive parameter features include an amount of variability in a given vital sign 342 and 352, a slope for the given vital sign 344 and 354, a combination of the slope for the given vital sign and an amount of deviation from a baseline range for the given vital sign 346 and 356, and an amount of deviation from an expected level of the given vital sign 348 and 358 (these examples are not intended to be limiting). Finally, a configurable combination of model features and interaction terms 390 (e.g., configured by entry and/or selection by a user) may be used by the trained model to predict various outcomes 392. Example 1 below describes an example trained logistical regression model that uses heart rate and mean arterial blood pressure to predict cardio vascular (CV) intervention or death.

Returning to FIG. 1, in some embodiments, model component 24 is and/or includes a population level configuration component and a patient level configuration component. The population level configuration may allow users to flexibly configure the weights, thresholds, default values, and/or other information for sub-populations at a time. For example, a user may increase the default alerting threshold for the entire cardiac surgery unit. As another example, the population level configuration component may facilitate changing the default baseline blood pressure values for heart failure patients admitted to the medical ICU. As a further example, the patient level configuration component may allow for specific adaptation of the system parameters for a given patient.

In some embodiments, prediction component 26 is configured to receive the vital signs information for individual 12 and/or other individuals. Prediction component 26 is configured to receive the vital signs information from sensors 14 and/or other devices. Prediction component 26 is configured to process the vital signs information via the prediction model (e.g., by providing the vital signs information as input to the prediction model to cause the prediction model to process the vital signs information and generate one or more outputs based thereon). In some embodiments, the processing, via the prediction model, generates (i) a first prediction related to a clinical intervention need for the individual 12 (e.g., a risk score or other prediction), (ii) sub-predictions contributing the first prediction or to at least another one of the sub-predictions, and (iii) relatedness information indicating how the first prediction and the contributing sub-predictions are related. The contributing sub-predictions correspond to respective ones of the weighted predictive parameter features and/or other information. In some embodiments, the processing of the vital signs information includes causing linking of the first prediction and the contributing sub-predictions based on the relatedness information and/or other information. As an example, by generating the predictions for different outcomes, the aggregate risk can be used. For example, a patient may have a high risk for intubation and a moderate risk for cardiovascular resuscitation. Together, this patient would have higher overall risk than a patient who only has a high risk for intubation but low risk for cardiovascular resuscitation.

In some embodiments, presentation component 28 is configured to cause a presentation related to a clinical intervention need for individual 12. The presentation is caused to be provided on user interface 40 and/or other user interfaces based on the first prediction related to a clinical intervention need for the individual, the sub-predictions contributing to the first prediction or to at least another one of the sub-predictions, the relatedness information indicating how the first prediction and the contributing sub-predictions are related, the linking information (e.g., as described above), and/or other information. In some embodiments, the presentation comprises graphical or other representations of the first prediction and the contributing sub-predictions such that user selection related to the first prediction causes display of one or more of the contributing sub-predictions. As an example, when a user taps, clicks, holds, releases, hoover over, etc., a representation of the first prediction, one or more of the contributing sub-predictions may be displayed on the user interface (e.g., in lieu of the first prediction, along with the first prediction, etc.). In some embodiments, the prediction model (described above) comprises a cardiovascular prediction model, a respiratory prediction model, and/or other models. In such embodiments, the presentation related to the clinical intervention need for the individual is a combination of a cardiovascular indicator determined based on weighted predictive cardiovascular parameter features and a respiratory indicator determined based on weighted predictive respiratory parameter features. In some embodiments, presentation component 28 is configured such that the presentation related to a clinical intervention need for individual 12 is a presentation of a risk score or other information. The risk score may be indicative of a need for acute intervention and/or other information.

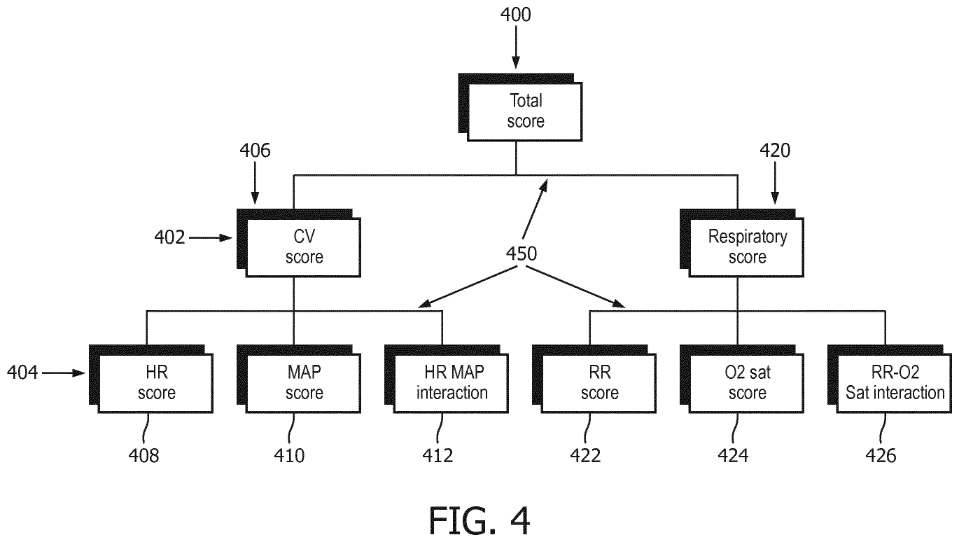

By way of a non-limiting example, FIG. 4 illustrates a risk score 400 presented to caregivers and/or other users that indicates a clinical intervention need for individual 12 (FIG. 1). FIG. 4 illustrates a layer-based presentation, but this is not intended to be limiting. In the example shown in FIG. 4, the presentation (e.g., risk score 400 comprises the first prediction (risk score 400) in a first layer and the contributing sub-predictions 402 and 404 in second and third layers (the illustration of three layers is not intended to be limiting). In the example shown in FIG. 4, the prediction model (described above) comprises a cardiovascular prediction model, and a respiratory prediction model. In this example, the presentation related to the clinical intervention need for the individual (risk score 400) is a combination of a cardiovascular (CV) indicator (score) 406 determined based on weighted predictive cardiovascular parameter features (heart rate (HR) score 408, mean arterial pressure (MAP) score 410, HR-MAP interaction 412) and a respiratory indicator (score) 420 determined based on weighted predictive respiratory parameter features (respiratory rate (RR) score 422, oxygen (O2) saturation (Sat) score 424, RR-O2 Sat interaction score 426). As shown in FIG. 4, the relatedness information indicating how the first prediction (risk score 400) and the contributing sub-predictions (406-426) and the linking between such contributors to the overall score are illustrated by connecting lines 450.

In some embodiments, individual 12 (FIG. 1) comprises a plurality of individuals (as described above), and presentation component 28 (FIG. 1) is configured to cause display of risk scores for the plurality of individuals. In some embodiments, presentation component 28 is configured to generate an ordered display representative of the plurality of individuals based on risk scores associated with the plurality of individuals. In such embodiments, presentation component 28 may be configured such that individuals 12 are ranked by the total score produced from the model (e.g., risk score 400), but presentation component 28 facilitates easy visualization of the second (402), third (406), and even fourth (not shown) layers of features contributing to risk. The fourth layer may be comprised, for example, of the core model which describes how individual scores (e.g., HR Score 408) was calculated in a clinically meaningful way. By way of a non-limiting example, Example 2 below shows example calculations and/or categorization of HR Score 408 and MAP Score 410, deviation from baseline (e.g., as shown in FIG. 2B), and deviation from (e.g., linear—this is not intended to be limiting—and/or other) projection covariates (e.g., as shown in FIG. 2C) using a predictive model for a given patient. The system allows for a large number of unique categories. If each of the 6 example parameters are classified into 5 categories and modeled independently, there would be 30 coefficients/weights. If two of these terms are interacted, there would be 45 coefficients (5×5=25 plus the other 4 parameters of 5 categories each). If three terms are interacted, there would be 140 coefficients (125 plus 15). If all 6 terms are interacted, there would be 15,625 unique combinations.

In some embodiments, presentation component 28 is configured such that user selection related to the first prediction (risk score 400) causes display of one or more of the contributing sub-predictions (e.g., the scores in layers 402, 404). For example, initially, only risk score 400 may be displayed by presentation component 28 on a touch screen of a computing device 18 (FIG. 1). Responsive to a caregiver and/or other users touching risk score 400 on the touch screen, presentation component 28 may cause the display to expand to show layer 402. Responsive to a similar touch on one of the sub-predictions in layer 402 (e.g., scores 406 and/or 420), presentation component may cause the display to expand to show layer 404, and so on.

Presentation component 28 is configured to expand the display to shown one or more given sub-predictions based on the relatedness information and/or the linking determined by prediction component 26 (FIG. 1) and/or other information. For example, in this example, prediction component 26 determined that HR score 408, MAP score 410, and HR-MAP interaction 412 are related to, and should be linked with, CV score 406. Scores 406, 422, 424, and 426 are also similarly related and linked.

In some embodiments, presentation component 28 is configured such that users can change the baseline values and/or weights for a given patient. For example, if a patient is known to have a high baseline heart rate due to an arrhythmia, the user can increase the baseline range for heart rate for that patient. This would decrease estimated risk for the patient with a high heart rate for the general population but relatively normal for this patient. In some embodiments, presentation component 28 may be configured such that users can change the threshold to alert for a given patient. This would allow users to acknowledge a patient has high risk but may not want to be notified unless their risk increases to a certain level.

Presentation component 28 is configured such that a user may similarly collapse the display back to risk score 400. In some embodiments, presentation component 28 is configured such that expanding display layers does not require user selection. For example, presentation component 28 may be configured such that the touch screen automatically displays risk score 400 (the first prediction) initially and then automatically displays layers 402 and 404 (the contributing sub-predictions) over time (e.g., based on their hierarchy and/or how they contribute to the first prediction), and/or for other reasons.

Using these clinically interpretable scores (for example), caregivers may easily understand why an individual 12 (FIG. 1) has a particular risk score and why the risk of the individual is higher or lower than another individual 12. In addition, system 10 (FIG. 1) may be configured to recalibrate a given model for different subgroups of patients to enhance an accuracy and/or meaningfulness of scores (described below). For example, a set of coefficients (weights) and/or population average values may differ for CABG patients than for patients admitted with heart failure. As such, the probability of needing an acute intervention may differ for two patients of these two different clinical subtypes (e.g., CABG patients versus heart failure patients). These subtypes may vary based on more than just admission diagnosis. Other subtypes may be based on age, ICU type, length of stay, etc., as well as combinations of subtypes (e.g., older heart failure patients versus younger heart failure patients, etc.).

Again, it should be emphasized that FIG. 4 is one example of many possible examples, and it is not intended to be limiting. For example, FIG. 4 could have been drawn to be more generic to represent the HR and MAP values along with other parameters yet to be determined.

Returning to FIG. 1, in some embodiments, model component 24, prediction component 26, presentation component 28, and/or other components of processor 20 and/or system 10 are configured to continuously provide additional patient information as input to the prediction model to further train the prediction model for generating predictions related to a need for clinical intervention for individual patients (e.g., by periodically providing the additional patient information, by automatically providing the additional patient information as it become available, or via other continuous techniques). The further training of the prediction model may cause the prediction model to develop an updated set of weighted predictive parameter features, for example. The updated set may be developed by one or more of (i) modification of the weighted predictive parameter features, (ii) removal of at least one of the weighted predictive parameter features from the prediction model, (iii) development of one or more additional weighted predictive parameter features, (iv) modifying interaction terms and/or categories, and/or other operations. In some embodiments, for example, prediction component 26 is configured to continuously receive, via one or more sensors 14, additional vital signs information for the one or more vital signs of individual 12 (e.g., by periodically obtaining the additional vital signs information, automatically obtaining the additional vital signs information as it become available, or via other continuous techniques). Prediction component 26 is configured to process, via the further-trained prediction model, the additional vital signs information to generate (i) an additional prediction related to a clinical intervention need for the individual, (ii) additional sub-predictions contributing to the additional prediction or to at least another one of the additional sub-predictions, (iii) additional relatedness information indicating how the additional prediction and the contributing additional sub-predictions are related, and/or other information. The contributing additional sub-predictions correspond to respective ones of the weighted predictive parameter features of the updated set. In some embodiments, model component 24, prediction component 26, presentation component 28, and/or other components are configured to cause linking of the additional prediction and the contributing additional sub-predictions based on the additional relatedness information; and update the information displayed on user interface 40 and/or other interfaces, based on the linking of the additional prediction and the contributing additional sub-predictions. The updated prediction (e.g., score) comprises the additional prediction and the additional contributing sub-predictions such that user selection related to the additional prediction causes display of one or more of the contributing additional sub-predictions.

Electronic storage 50 comprises electronic storage media that electronically stores information. The electronic storage media of electronic storage 50 may comprise one or both of system storage that is provided integrally (i.e., substantially non-removable) with system 10 and/or removable storage that is removably connectable to system 10 via, for example, a port (e.g., a USB port, a firewire port, etc.) or a drive (e.g., a disk drive, etc.). Electronic storage 50 may be (in whole or in part) a separate component within system 10, or electronic storage 50 may be provided (in whole or in part) integrally with one or more other components of system 10 (e.g., computing devices 18, processor 20, etc.). In some embodiments, electronic storage 50 may be located in a server together with processor 20, in a server that is part of external resources 16, in a computing device 18, and/or in other locations. Electronic storage 50 may comprise one or more of optically readable storage media (e.g., optical disks, etc.), magnetically readable storage media (e.g., magnetic tape, magnetic hard drive, floppy drive, etc.), electrical charge-based storage media (e.g., EPROM, RAM, etc.), solid-state storage media (e.g., flash drive, etc.), and/or other electronically readable storage media. Electronic storage 50 may store software algorithms, information determined by processor 20, information received via a computing device 18 and/or graphical user interface 40 and/or other external computing systems, information received from external resources 16, information received from sensors 14, and/or other information that enables system 10 to function as described herein.

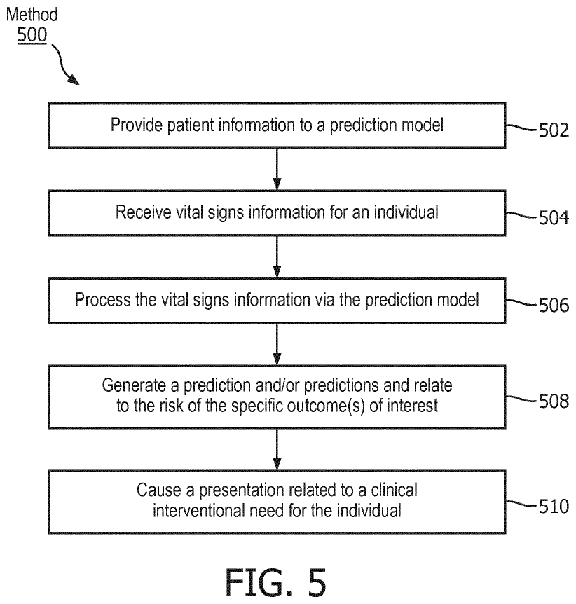

FIG. 5 illustrates a method 500 for providing a layer-based presentation of a model-generated patient-related prediction with a prediction system. The system comprises one or more hardware processors and/or other components. The one or more hardware processors are configured by machine readable instructions to execute computer program components. The computer program components include a patient information component, a model component, a prediction component, a presentation component, and/or other components. The operations of method 500 presented below are intended to be illustrative. In some embodiments, method 500 may be accomplished with one or more additional operations not described, and/or without one or more of the operations discussed. Additionally, the order in which the operations of method 500 are illustrated in FIG. 5 and described below is not intended to be limiting.

In some embodiments, method 500 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 500 in response to instructions stored electronically on an electronic storage medium. The one or more processing devices may include one or more devices configured through hardware, firmware, and/or software to be specifically designed for execution of one or more of the operations of method 500.

At an operation 502, patient information is provided to a prediction model. The patient information is provided to the prediction model to train the prediction model for generating predictions related to a need for clinical intervention for individual patients and/or for other purposes. The training of the prediction model causes the prediction model to develop weighted predictive parameter features that correspond to patient vital signs and/or other parameters. In some embodiments, the weighted predictive parameter features include an amount of variability in a given vital sign, a slope for the given vital sign determined based on values of the given vital sign over time, a combination of the slope for the given vital sign and an amount of deviation from a baseline range for the given vital sign, a combination of an amount of deviation from an expected level of the given vital sign for the monitored individual, and the amount of deviation from the baseline range for the given vital sign, and/or other features. The patient information comprises initial vital signs of patients, treatments provided to the patients with the respective initial vital signs, respective vital signs resulting from the treatments, and/or other information. In some embodiments, operation 502 is performed by a processor component the same as or similar to patient information component 22 and/or model component 24 (shown in FIG. 1 and described herein).

At an operation 504, vital signs information for an individual is received. In some embodiments, the vital signs information is received from one or more sensors operatively coupled to the individual. In some embodiments, the one or more sensors include at least one cardiovascular sensor and one respiratory sensor. In some embodiments, operation 504 is performed by a processor component the same as or similar to prediction component 26 (shown in FIG. 1 and described herein).

At an operation 506, the vital signs information is processed via the prediction model. The processing, via the trained prediction model, generates (i) a first prediction related a clinical intervention need for the individual, (ii) sub predictions contributing the first prediction or to at least another one of the sub-predictions, and (iii) relatedness information indicating how the first prediction and the contributing sub-predictions are related. The contributing sub-predictions correspond to respective ones of the weighted predictive parameter features. Processing also includes causing linking of the first prediction and the contributing sub-predictions based on the relatedness information and/or other information. In some embodiments, operation 506 is performed by a processor component the same as or similar to prediction component 26 (shown in FIG. 1 and described herein).

At an operation 508, a prediction and/or predictions related to a clinical interventional need are generated. In some embodiments, the prediction model (described above) comprises a cardiovascular prediction model, a respiratory prediction model, and/or other models. In such embodiments, the prediction related to the clinical intervention need for the individual is a combination of a cardiovascular indicator determined based on weighted predictive cardiovascular parameter features and a respiratory indicator determined based on weighted predictive respiratory parameter features. As described above, these features may be weighted in such a way so as to cause the prediction to be indicative of one or more specific outcomes of interest. In some embodiments, operation 508 is performed by a processor component the same as or similar to prediction component 26 (shown in FIG. 1 and described herein).

At an operation 510, a presentation related to a clinical intervention need for the individual is caused. The presentation is caused, via a user interface, based on the linking and/or other information. The presentation comprises the first prediction and the contributing sub-predictions such that user selection related to the first prediction causes display of one or more of the contributing sub-predictions. In some embodiments, the presentation related to a clinical intervention need for the individual is a risk score. The risk score may be indicative of a need for acute intervention and/or other information. In some embodiments, the individual (described above) comprises a plurality of individuals, and operation 510 includes displaying risk scores for the plurality of individuals. In some embodiments, operation 510 includes generating an ordered display representative of the plurality of individuals based on risk scores associated with the plurality of individuals. In some embodiments, operation 510 is performed by a processor component the same as or similar to presentation component 28 (shown in FIG. 1 and described herein).

In some embodiments, method 500 comprises continuously providing additional patient information as input to the prediction model to further train the prediction model for generating predictions related to a need for clinical intervention for individual patients. The further training of the prediction model may cause the prediction model to develop an updated set of weighted predictive parameter features, for example. The updated set may be developed by one or more of (i) modification of the weighted predictive parameter features, (ii) removal of at least one of the weighted predictive parameter features from the prediction model, (iii) development of one or more additional weighted predictive parameter features, (iv) modifying interaction terms and/or categories, and/or other operations. In some embodiments, method 500 comprises receiving (e.g., continuously and/or intermittently), via the one or more sensors, additional vital signs information for the one or more vital signs of the individual; and processing, via the further-trained prediction model, the additional vital signs information to generate (i) an additional prediction related to a clinical intervention need for the individual, (ii) additional sub-predictions contributing to the additional prediction or to at least another one of the additional sub-predictions, (iii) additional relatedness information indicating how the additional prediction and the contributing additional sub-predictions are related, and/or other information. The contributing additional sub-predictions correspond to respective ones of the weighted predictive parameter features of the updated set. In some embodiments, method 500 includes causing linking of the additional prediction and the contributing additional sub-predictions based on the additional relatedness information; and updating the user interface, based on the linking of the additional prediction and the contributing additional sub-predictions. The updated prediction comprises the additional prediction and the additional contributing sub-predictions such that user selection related to the additional prediction causes display of one or more of the contributing additional sub-predictions.

### EXAMPLES

### Example 1

Logistic Regression Model of Heart Rate and Mean Arterial Blood
Pressure Data Predicting CV Intervention or Death (n = 70,000).

| | | | 95% |
|---|---|---|---|
| | Odds | P- | Confidence |
| Variables | Ratio | value | Interval |
| hr_var1 | 3.642 × | 0.000 | 0.000, 0.000 |
| | 10⁻⁷ | | |
| hr_var2 | 1 | . | 1.000, 1.000 |
| hr_var3 | 64.723 | 0.000 | 61.079, 68.585 |
| map_var1 | 2.561 × | 0.000 | 7.0 × 10⁶, |
| | 10⁷ | | 9.3 × 10⁶7 |
| map_var2 | 49.336 | 0.000 | 43.520, 55.929 |
| map_var3 | 9.791 | 0.000 | 9.518, 10.072 |
| 0.hr_slope_cat#0.hr_dpm_cat | Ref | Ref | Ref |
| 0.hr_slope_cat#1.hr_dpm_cat | 0.716 | 0.010 | 0.557, 0.920 |
| 0.hr_slope_cat#2.hr_dpm_cat | 0.469 | 0.000 | 0.356, 0.619 |
| 0.hr_slope_cat#8.hr_dpm_cat | 2.378 | 0.000 | 2.262, 2.499 |
| 0.hr_slope_cat#9.hr_dpm_cat | 4.617 | 0.000 | 4.381, 4.866 |
| 1.hr_slope_cat#0.hr_dpm_cat | 2.019 | 0.000 | 1.919, 2.124 |
| 1.hr_slope_cat#1.hr_dpm_cat | 4.938 | 0.000 | 3.827, 6.373 |
| 1.hr_slope_cat#2.hr_dpm_cat | 1.449 | 0.011 | 1.095, 1.917 |
| 1.hr_slope_cat#8.hr_dpm_cat | 4.685 | 0.000 | 4.363, 5.031 |
| 1.hr_slope_cat#9.hr_dpm_cat | 6.621 | 0.000 | 6.067, 7.226 |
| 2.hr_slope_cat#0.hr_dpm_cat | 1.472 | 0.000 | 1.404, 1.544 |
| 2.hr_slope_cat#1.hr_dpm_cat | 1.981 | 0.000 | 1.529, 2.566 |
| 2.hr_slope_cat#2.hr_dpm_cat | 0.855 | 0.278 | 0.646, 1.133 |
| 2.hr_slope_cat#8.hr_dpm_cat | 3.476 | 0.000 | 3.244, 3.725 |
| 2.hr_slope_cat#9.hr_dpm_cat | 6.240 | 0.000 | 5.764, 6.755 |
| 8.hr_slope_cat#0.hr_dpm_cat | 1.131 | 0.000 | 1.079, 1.186 |
| 8.hr_slope_cat#1.hr_dpm_cat | 0.422 | 0.000 | 0.312, 0.570 |
| 8.hr_slope_cat#2.hr_dpm_cat | 0.388 | 0.000 | 0.291, 0.517 |
| 8.hr_slope_cat#8.hr_dpm_cat | 2.597 | 0.000 | 2.428, 2.779 |
| 8.hr_slope_cat#9.hr_dpm_cat | 5.207 | 0.000 | 4.849, 5.591 |
| 9.hr_slope_cat#0.hr_dpm_cat | 1.258 | 0.000 | 1.206, 1.312 |
| 9.hr_slope_cat#1.hr_dpm_cat | 0.480 | 0.000 | 0.353, 0.654 |
| 9.hr_slope_cat#2.hr_dpm_cat | 0.256 | 0.000 | 0.190, 0.345 |
| 9.hr_slope_cat#8.hr_dpm_cat | 3.149 | 0.000 | 2.962, 3.349 |
| 9.hr_slope_cat#9.hr_dpm_cat | 4.528 | 0.000 | 4.260, 4.812 |
| 0.hr_dlp_cat#0.hr_dpm_cat | Ref | Ref | Ref |
| 0.hr_dlp_cat#1.hr_dpm_cat | 1.136 | 0.320 | 0.884, 1.460 |
| 0.hr_dlp_cat#2.hr_dpm_cat | 1.469 | 0.007 | 1.115, 1.937 |
| 0.hr_dlp_cat#8.hr_dpm_cat | 0.772 | 0.000 | 0.735, 0.812 |
| 0.hr_dlp_cat#9.hr_dpm_cat | 0.942 | 0.029 | 0.893, 0.993 |
| 1.hr_dlp_cat#0.hr_dpm_cat | 2.454 | 0.000 | 2.386, 2.524 |
| 1.hr_dlp_cat#1.hr_dpm_cat | 7.698 | 0.000 | 5.994, 9.887 |
| 1.hr_dlp_cat#2.hr_dpm_cat | 9.140 | 0.000 | 6.921, 12.069 |
| 1.hr_dlp_cat#8.hr_dpm_cat | 1.211 | 0.000 | 1.139, 1.288 |
| 1.hr_dlp_cat#9.hr_dpm_cat | 1.050 | 0.282 | 0.961, 1.147 |
| 2.hr_dlp_cat#0.hr_dpm_cat | 1.048 | 0.000 | 1.040, 1.056 |
| 2.hr_dlp_cat#1.hr_dpm_cat | 1.842 | 0.000 | 1.431, 2.369 |
| 2.hr_dlp_cat#2.hr_dpm_cat | 1.696 | 0.000 | 1.287, 2.236 |
| 2.hr_dlp_cat#8.hr_dpm_cat | 0.842 | 0.000 | 0.801, 0.885 |
| 2.hr_dlp_cat#9.hr_dpm_cat | 1.083 | 0.004 | 1.027, 1.143 |
| 8.hr_dlp_cat#0.hr_dpm_cat | 0.975 | 0.000 | 0.967, 0.983 |
| 8.hr_dlp_cat#1.hr_dpm_cat | 0.952 | 0.702 | 0.740, 1.225 |
| 8.hr_dlp_cat#2.hr_dpm_cat | 1.473 | 0.007 | 1.118, 1.942 |
| 8.hr_dlp_cat#8.hr_dpm_cat | 0.757 | 0.000 | 0.720, 0.795 |
| 8.hr_dlp_cat#9.hr_dpm_cat | 0.942 | 0.030 | 0.893, 0.994 |
| 9.hr_dlp_cat#0.hr_dpm_cat | 1.264 | 0.000 | 1.170, 1.365 |
| 9.hr_dlp_cat#1.hr_dpm_cat | 1 | . | 1.000, 1.000 |
| 9.hr_dlp_cat#2.hr_dpm_cat | 1 | . | 1.000, 1.000 |
| 9.hr_dlp_cat#8.hr_dpm_cat | 1 | . | 1.000, 1.000 |
| 9.hr_dlp_cat#9.hr_dpm_cat | 1 | . | 1.000, 1.000 |
| 0.map_slope_cat#0.map_dpm_cat | Ref | Ref | Ref |
| 0.map_slope_cat#1.map_dpm_cat | 4.580 | 0.000 | 4.215, 4.975 |
| 0.map_slope_cat#2.map_dpm_cat | 1.674 | 0.000 | 1.555, 1.803 |
| 0.map_slope_cat#8.map_dpm_cat | 0.661 | 0.000 | 0.634, 0.689 |
| 0.map_slope_cat#9.map_dpm_cat | 0.920 | 0.036 | 0.852, 0.994 |
| 1.map_slope_cat#0.map_dpm_cat | 1.271 | 0.000 | 1.218, 1.326 |
| 1.map_slope_cat#1.map_dpm_cat | 4.954 | 0.000 | 4.520, 5.429 |
| 1.map_slope_cat#2.map_dpm_cat | 1.780 | 0.000 | 1.636, 1.936 |
| 1.map_slope_cat#8.map_dpm_cat | 0.893 | 0.000 | 0.841, 0.947 |
| 1.map_slope_cat#9.map_dpm_cat | 1.234 | 0.000 | 1.130, 1.348 |
| 2.map_slope_cat#0.map_dpm_cat | 0.979 | 0.415 | 0.931, 1.030 |
| 2.map_slope_cat#1.map_dpm_cat | 4.617 | 0.000 | 4.192, 5.085 |
| 2.map_slope_cat#2.map_dpm_cat | 1.605 | 0.000 | 1.468, 1.754 |
| 2.map_slope_cat#8.map_dpm_cat | 0.671 | 0.000 | 0.627, 0.718 |
| 2.map_slope_cat#9.map_dpm_cat | 1.066 | 0.226 | 0.962, 1.182 |
| 8.map_slope_cat#0.map_dpm_cat | 1.040 | 0.113 | 0.991, 1.092 |
| 8.map_slope_cat#1.map_dpm_cat | 4.072 | 0.000 | 3.694, 4.487 |
| 8.map_slope_cat#2.map_dpm_cat | 1.870 | 0.000 | 1.711, 2.044 |
| 8.map_slope_cat#8.map_dpm_cat | 0.658 | 0.000 | 0.619, 0.701 |
| 8.map_slope_cat#9.map_dpm_cat | 0.929 | 0.084 | 0.855, 1.009 |
| 9.map_slope_cat#0.map_dpm_cat | 1.386 | 0.000 | 1.330, 1.443 |
| 9.map_slope_cat#1.map_dpm_cat | 5.294 | 0.000 | 4.815, 5.821 |
| 9.map_slope_cat#2.map_dpm_cat | 2.947 | 0.000 | 2.706, 3.210 |
| 9.map_slope_cat#8.map_dpm_cat | 0.820 | 0.000 | 0.778, 0.863 |
| 9.map_slope_cat#9.map_dpm_cat | 0.908 | 0.000 | 0.861, 0.957 |
| 0.map_dlp_cat#0.map_dpm_cat | Ref | Ref | Ref |
| 0.map_dlp_cat#1.map_dpm_cat | 1.535 | 0.000 | 1.415, 1.666 |
| 0.map_dlp_cat#2.map_dpm_cat | 1.512 | 0.000 | 1.405, 1.627 |
| 0.map_dlp_cat#8.map_dpm_cat | 0.757 | 0.000 | 0.730, 0.784 |
| 0.map_dlp_cat#9.map_dpm_cat | 0.639 | 0.000 | 0.605, 0.674 |
| 1.map_dlp_cat#0.map_dpm_cat | 0.442 | 0.000 | 0.435, 0.449 |
| 1.map_dlp_cat#1.map_dpm_cat | 0.914 | 0.034 | 0.841, 0.992 |
| 1.map_dlp_cat#2.map_dpm_cat | 0.599 | 0.000 | 0.555, 0.646 |
| 1.map_dlp_cat#8.map_dpm_cat | 0.398 | 0.000 | 0.370, 0.427 |
| 1.map_dlp_cat#9.map_dpm_cat | 0.430 | 0.000 | 0.367, 0.503 |
| 2.map_dlp_cat#0.map_dpm_cat | 0.766 | 0.000 | 0.759, 0.772 |
| 2.map_dlp_cat#1.map_dpm_cat | 1.388 | 0.000 | 1.279, 1.508 |
| 2.map_dlp_cat#2.map_dpm_cat | 1.112 | 0.006 | 1.033, 1.197 |
| 2.map_dlp_cat#8.map_dpm_cat | 0.631 | 0.000 | 0.604, 0.659 |
| 2.map_dlp_cat#9.map_dpm_cat | 0.458 | 0.000 | 0.413, 0.507 |
| 8.map_dlp_cat#0.map_dpm_cat | 0.954 | 0.000 | 0.947, 0.961 |
| 8.map_dlp_cat#1.map_dpm_cat | 1.907 | 0.000 | 1.756, 2.072 |
| 8.map_dlp_cat#2.map_dpm_cat | 1.569 | 0.000 | 1.457, 1.689 |
| 8.map_dlp_cat#8.map_dpm_cat | 0.774 | 0.000 | 0.747, 0.803 |
| 8.map_dlp_cat#9.map_dpm_cat | 0.623 | 0.000 | 0.591, 0.657 |
| 9.map_dlp_cat#0.map_dpm_cat | 0.739 | 0.000 | 0.723, 0.756 |
| 9.map_dlp_cat#1.map_dpm_cat | 1 | . | 1.000, 1.000 |
| 9.map_dlp_cat#2.map_dpm_cat | 1 | . | 1.000, 1.000 |
| 9.map_dlp_cat#8.map_dpm_cat | 1 | . | 1.000, 1.000 |
| 9.map_dlp_cat#9.map_dpm_cat | 1 | . | 1.000, 1.000 |
| 0.hr_slope_cat#0.map_slope_cat | Ref | Ref | Ref |
| 0.hr_slope_cat#1.map_slope_cat | 0.659 | 0.000 | 0.632, 0.688 |
| 0.hr_slope_cat#2.map_slope_cat | 0.931 | 0.007 | 0.885, 0.979 |
| 0.hr_slope_cat#8.map_slope_cat | 0.974 | 0.286 | 0.927, 1.022 |
| 0.hr_slope_cat#9.map_slope_cat | 0.843 | 0.000 | 0.809, 0.878 |
| 1.hr_slope_cat#0.map_slope_cat | 1.006 | 0.824 | 0.951, 1.065 |
| 1.hr_slope_cat#1.map_slope_cat | 0.999 | 0.969 | 0.954, 1.046 |
| 1.hr_slope_cat#2.map_slope_cat | 1.108 | 0.001 | 1.044, 1.174 |
| 1.hr_slope_cat#8.map_slope_cat | 0.903 | 0.002 | 0.849, 0.960 |
| 1.hr_slope_cat#9.map_slope_cat | 1 | . | 1.000, 1.000 |
| 2.hr_slope_cat#0.map_slope_cat | 0.999 | 0.976 | 0.949, 1.052 |
| 2.hr_slope_cat#1.map_slope_cat | 0.792 | 0.000 | 0.758, 0.827 |
| 2.hr_slope_cat#2.map_slope_cat | 1.032 | 0.250 | 0.978, 1.090 |
| 2.hr_slope_cat#8.map_slope_cat | 1.023 | 0.416 | 0.969, 1.080 |
| 2.hr_slope_cat#9.map_slope_cat | 1 | . | 1.000, 1.000 |
| 8.hr_slope_cat#0.map_slope_cat | 1.138 | 0.000 | 1.083, 1.197 |
| 8.hr_slope_cat#1.map_slope_cat | 0.928 | 0.001 | 0.887, 0.970 |
| 8.hr_slope_cat#2.map_slope_cat | 1.137 | 0.000 | 1.076, 1.201 |
| 8.hr_slope_cat#8.map_slope_cat | 1.139 | 0.000 | 1.081, 1.200 |
| 8.hr_slope_cat#9.map_slope_cat | 1 | . | 1.000, 1.000 |
| 9.hr_slope_cat#0.map_slope_cat | 1 | . | 1.000, 1.000 |
| 9.hr_slope_cat#1.map_slope_cat | 1 | . | 1.000, 1.000 |
| 9.hr_slope_cat#2.map_slope_cat | 1 | . | 1.000, 1.000 |
| 9.hr_slope_cat#8.map_slope_cat | 1 | . | 1.000, 1.000 |
| 9.hr_slope_cat#9.map_slope_cat | 1 | . | 1.000, 1.000 |

### Example 2

Criteria For Categorizing HR and MAP Slope, Deviation from Population Mean, and
Deviation from Linear Projection Covariates

| | HR | | | MAP | | |
|---|---|---|---|---|---|---|
| | | Deviation | Deviation | | Deviation | Deviation |
| | | from | from | | from | from |
| | Slope for HR | population | linear | Slope for MAP | population | linear |
| Category | (/min) | mean | projection | (/min) | mean | projection |
| 9 | ≥.33 | ≥30 | ≥20 | ≥.0625 | ≥20 | ≥20 |
| 8 | .167. to .32 | >0 to <30 | >0 to <20 | .02083 to | >0 to <20 | >0 to <20 |
| | | | | .0624 | | |
| 0 | −.168 to | 0 | 0 | −.02084 to | 0 | 0 |
| | .166 | | | .02082 | | |
| 2 | −.34 to | >−15 to <0 | >−10 to <0 | −.0626 to | >−10 to <0 | >−10 to <0 |
| | −.167 | | | −.02083 | | |
| 1 | ≤−.33 | ≤−15 | ≤−10 | ≤−.0625 | ≤−10 | ≤−10 |

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

1. A system configured to provide a layer-based presentation of a model-generated patient-related prediction, the system comprising one or more hardware processors configured by machine readable instructions to:
provide patient information as input to a prediction model to train the prediction model for generating predictions related to a need for clinical intervention for individual patients, the training of the prediction model causing the prediction model to develop weighted predictive parameter features that correspond to patient vital signs, patient demographic information, patient physiology, patient laboratory data, a patient diagnosis, or patient treatment data, the patient information comprising initial vital signs of patients, treatments provided to the patients with the respective initial vital signs, and respective vital signs resulting from the treatments;
receive, via one or more sensors, vital signs information for one or more vital signs of an individual;
process, via the trained prediction model, the received information to generate (i) a first prediction related a clinical intervention need for the individual, (ii) sub-predictions contributing the first prediction or to at least another one of the sub-predictions, and (iii) relatedness information indicating how the first prediction and the contributing sub-predictions are related, the contributing sub-predictions corresponding to respective ones of the weighted predictive parameter features;
cause linking of the first prediction and the contributing sub-predictions based on the relatedness information; and
cause, via a user interface, based on the linking, a presentation related to a clinical intervention need for the individual, the presentation comprising the first prediction and the contributing sub-predictions such that user selection related to the first prediction causes display of one or more of the contributing sub-predictions,
wherein the prediction model comprises a cardiovascular prediction model and a respiratory prediction model and wherein the presentation related to the clinical intervention need for the individual is configured such that the first prediction is a combination of a cardiovascular indicator sub-prediction determined based on weighted predictive cardiovascular parameter features and a respiratory indicator sub-prediction determined based on weighted predictive respiratory parameter features.

2. The system of claim 1, wherein the one or more hardware processors are configured to:
intermittently, in a retrospective fashion, provide additional patient information as input to the prediction model to further train the prediction model for generating predictions related to a need for clinical intervention for individual patients, the further training of the prediction model causing the prediction model to develop an updated set of weighted predictive parameter features, the updated set being developed by one or more of (i) modification of the weighted predictive parameter features, (ii) removal of at least one of the weighted predictive parameter features from the prediction model, or (iii) development of one or more additional weighted predictive parameter features;
receive, via the one or more sensors, additional vital signs information for the one or more vital signs of the individual;
process, via the further-trained prediction model, the additional vital signs information to generate (i) an additional prediction related to a clinical intervention need for the individual, (ii) additional sub-predictions contributing to the additional prediction or to at least another one of the additional sub-predictions, (iii) additional relatedness information indicating how the additional prediction and the contributing additional sub-predictions are related, the contributing additional sub-predictions corresponding to respective ones of the weighted predictive parameter features of the updated set;
cause linking of the additional prediction and the contributing additional sub-predictions based on the additional relatedness information; and
update, via the user interface, based on the linking of the additional prediction and the contributing additional sub-predictions, the presentation related to a clinical intervention need for the individual, the updated prediction comprising the additional prediction and the additional contributing sub-predictions such that user selection related to the additional prediction causes display of one or more of the contributing additional sub-predictions.

3. The system of claim 1, wherein the one or more hardware processors are configured such that the weighted predictive parameter features include one or more of:
an amount of variability in a given vital sign;
a slope for the given vital sign determined based on values of the given vital sign over time;
an amount of deviation from a baseline for the given vital sign; or
an amount of deviation from an expected level of the given vital sign for the individual.

4. The system of claim 1, wherein the one or more hardware processors are configured such that the presentation related to a clinical intervention need for the individual is a risk score, the risk score indicative of a need for acute intervention, wherein the individual is continuously monitored via the one or more sensors, and wherein the risk score is continuously updated.

5. The system of claim 4, wherein the one or more hardware processors are configured such that the individual comprises a plurality of individuals; wherein the one or more hardware processors are further configured to generate an ordered display representative of the plurality of individuals based on risk scores associated with the plurality of individuals.

6. The system of claim 1, wherein the one or more hardware processors are configured such that the prediction model is derived from a logistical regression statistical model.

7. The system of claim 1, further comprising the one or more sensors configured to generate output signals that convey the vital signs information, the one or more sensors comprising a cardiovascular sensor and a respiratory sensor operatively coupled to the individual.

8. (canceled)

9. A method for providing a layer-based presentation of a model-generated patient-related prediction with a prediction system, the system comprising one or more hardware processors configured by machine readable instructions, the method comprising:
providing patient information as input to a prediction model to train the prediction model for generating predictions related to a need for clinical intervention for individual patients, the training of the prediction model causing the prediction model to develop weighted predictive parameter features that correspond to patient vital signs, patient demographic information, patient physiology, patient laboratory data, a patient diagnosis, or patient treatment data, the patient information comprising initial vital signs of patients, treatments provided to the patients with the respective initial vital signs, and respective vital signs resulting from the treatments;
receiving, via one or more sensors, vital signs information for one or more vital signs of an individual;
processing, via the trained prediction model, the received information to generate (i) a first prediction related a clinical intervention need for the individual, (ii) sub-predictions contributing the first prediction or to at least another one of the sub-predictions, and (iii) relatedness information indicating how the first prediction and the contributing sub-predictions are related, the contributing sub-predictions corresponding to respective ones of the weighted predictive parameter features;
causing linking of the first prediction and the contributing sub-predictions based on the relatedness information; and
causing, via a user interface, based on the linking, a presentation related to a clinical intervention need for the individual, the presentation comprising the first prediction and the contributing sub-predictions such that user selection related to the first prediction causes display of one or more of the contributing sub-predictions,
wherein the prediction model comprises a cardiovascular prediction model and a respiratory prediction model and wherein the presentation related to the clinical intervention need for the individual is configured such that the first prediction is a combination of a cardiovascular indicator sub-prediction determined based on weighted predictive cardiovascular parameter features and a respiratory indicator sub-prediction determined based on weighted predictive respiratory parameter features.

10. The method of claim 9, further comprising:
intermittently, in a retrospective fashion, providing additional patient information as input to the prediction model to further train the prediction model for generating predictions related to a need for clinical intervention for individual patients, the further training of the prediction model causing the prediction model to develop an updated set of weighted predictive parameter features, the updated set being developed by one or more of (i) modification of the weighted predictive parameter features, (ii) removal of at least one of the weighted predictive parameter features from the prediction model, or (iii) development of one or more additional weighted predictive parameter features;
receiving, via the one or more sensors, additional vital signs information for the one or more vital signs of the individual;
processing, via the further-trained prediction model, the additional vital signs information to generate (i) an additional prediction related to a clinical intervention need for the individual, (ii) additional sub-predictions contributing to the additional prediction or to at least another one of the additional sub-predictions, (iii) additional relatedness information indicating how the additional prediction and the contributing additional sub-predictions are related, the contributing additional sub-predictions corresponding to respective ones of the weighted predictive parameter features of the updated set;
causing linking of the additional prediction and the contributing additional sub-predictions based on the additional relatedness information; and
updating, via the user interface, based on the linking of the additional prediction and the contributing additional sub-predictions, the presentation related to a clinical intervention need for the individual, the updated prediction comprising the additional prediction and the additional contributing sub-predictions such that user selection related to the additional prediction causes display of one or more of the contributing additional sub-predictions.

11. The method of claim 9, wherein the weighted predictive parameter features include one or more of:
an amount of variability in a given vital sign;
a slope for the given vital sign determined based on values of the given vital sign over time;
an amount of deviation from a baseline for the given vital sign; or
an amount of deviation from an expected level of the given vital sign for the individual.

12. The method of claim 9, wherein the presentation related to a clinical intervention need for the individual is a risk score, the risk score indicative of a need for acute intervention, wherein the individual is continuously monitored via the one or more sensors, and wherein the risk score is continuously updated.

13. The method of claim 12, wherein the individual comprises a plurality of individuals; the method further comprising generating an ordered display representative of the plurality of individuals based on risk scores associated with the plurality of individuals.

14. The method of claim 9, further comprising generating, with the one or more sensors, output signals that convey the vital signs information, the one or more sensors comprising a cardiovascular sensor and a respiratory sensor operatively coupled to the individual.

15. (canceled)

16. A system configured to provide a layer-based presentation of a model-generated patient-related prediction, the system comprising:
means for providing patient information as input to a prediction model to train the prediction model for generating predictions related to a need for clinical intervention for individual patients, the training of the prediction model causing the prediction model to develop weighted predictive parameter features that correspond to patient vital signs, patient demographic information, patient physiology, patient laboratory data, a patient diagnosis, or patient treatment data, the patient information comprising initial vital signs of patients, treatments provided to the patients with the respective initial vital signs, and respective vital signs resulting from the treatments;
means for receiving, via one or more sensors, vital signs information for one or more vital signs of an individual;
means for processing, via the trained prediction model, the received information to generate (i) a first prediction related a clinical intervention need for the individual, (ii) sub-predictions contributing the first prediction or to at least another one of the sub-predictions, and (iii) relatedness information indicating how the first prediction and the contributing sub-predictions are related, the contributing sub-predictions corresponding to respective ones of the weighted predictive parameter features;
means for causing linking of the first prediction and the contributing sub-predictions based on the relatedness information; and
means for causing, via a user interface, based on the linking, a presentation related to a clinical intervention need for the individual, the presentation comprising the first prediction and the contributing sub-predictions such that user selection related to the first prediction causes display of one or more of the contributing sub-predictions,
wherein the prediction model comprises a cardiovascular prediction model and a respiratory prediction model and wherein the presentation related to the clinical intervention need for the individual is configured such that the first prediction is a combination of a cardiovascular indicator sub-prediction determined based on weighted predictive cardiovascular parameter features and a respiratory indicator sub-prediction determined based on weighted predictive respiratory parameter features.

17. The system of claim 16, further comprising:
means for intermittently, in a retrospective fashion, providing additional patient information as input to the prediction model to further train the prediction model for generating predictions related to a need for clinical intervention for individual patients, the further training of the prediction model causing the prediction model to develop an updated set of weighted predictive parameter features, the updated set being developed by one or more of (i) modification of the weighted predictive parameter features, (ii) removal of at least one of the weighted predictive parameter features from the prediction model, or (iii) development of one or more additional weighted predictive parameter features;
means for receiving, via the one or more sensors, additional vital signs information for the one or more vital signs of the individual;
means for processing, via the further-trained prediction model, the additional vital signs information to generate (i) an additional prediction related to a clinical intervention need for the individual, (ii) additional sub-predictions contributing to the additional prediction or to at least another one of the additional sub-predictions, (iii) additional relatedness information indicating how the additional prediction and the contributing additional sub-predictions are related, the contributing additional sub-predictions corresponding to respective ones of the weighted predictive parameter features of the updated set;
means for causing linking of the additional prediction and the contributing additional sub-predictions based on the additional relatedness information; and
means for updating, via the user interface, based on the linking of the additional prediction and the contributing additional sub-predictions, the presentation related to a clinical intervention need for the individual, the updated prediction comprising the additional prediction and the additional contributing sub-predictions such that user selection related to the additional prediction causes display of one or more of the contributing additional sub-predictions.

18. The system of claim 16, wherein the weighted predictive parameter features include one or more of:
an amount of variability in a given vital sign;
a slope for the given vital sign determined based on values of the given vital sign over time;
an amount of deviation from a baseline for the given vital sign; or
an amount of deviation from an expected level of the given vital sign for the individual.

19. The system of claim 16, wherein the presentation related to a clinical intervention need for the individual is a risk score, the risk score indicative of a need for acute intervention, wherein the individual is continuously monitored via the one or more sensors, and wherein the risk score is continuously updated.

20. The system of claim 16, further comprising the one or more sensors, the one or more sensors configured to generate output signals that convey the vital signs information, the one or more sensors comprising a cardiovascular sensor and a respiratory sensor operatively coupled to the individual.
